(19) United States
(12) Patent Application Publication
(10) Pub. No.: US 2024/0122553 A1
(43) Pub. Date: Apr. 18, 2024

(54) METHOD FOR USING BRAINWAVE AUSCULTATOR

(71) Applicant: National Taipei University of Technology, Taipei (TW)

(72) Inventors: Ren-Guey LEE, Taipei (TW); Tei-Wei HUNG, Taipei (TW)

(73) Assignee: National Taipei University of Technology, Taipei (TW)

(21) Appl. No.: 17/968,382

(22) Filed: Oct. 18, 2022

(51) Int. Cl. A61B 5/00; A61B 5/386

(52) U.S. Cl. CPC A61B 5/7415; A61B 5/386

(57) ABSTRACT

A brainwave auscultation method includes steps: providing a brainwave auscultation device; placing a brainwave pickup unit of the brainwave auscultation device on the head of a testee to acquire a primitive brainwave signal of the testee, and transmitting the primitive brainwave signal to a signal processing unit; the signal processing unit filtering the primitive brainwave signal according to a waveband reservation standard to generate a preparatory signal, wherein wavebands reserved by the waveband reservation standard include a δ waveband, a θ waveband, an α waveband, a β waveband, and a γ waveband; the signal processing unit shifting a central frequency of the preparatory signal to an audible range of human ears; the signal processing unit performing spread-spectrum operation to the shifted preparatory signal to generate a pre-vocalization signal whose frequencies range from 20 Hz to 20 kHz; and making a loudspeaker generate sounds based on the pre-vocalization signal.

### BACKGROUND OF THE INVENTION

### Field of the Invention

The present invention relates to a brainwave auscultation method, particularly a brainwave auscultation method that enables the physician to diagnose brainwaves with auditory sensation instantly.

### Description of the Prior Art

With advance of medical technology, brainwave inspection has become a common diagnosis measure. A brain contains billions of neurons joined closely and interconnecting through electrical signals, whereby humans can act, think, sleep, etc. A neurological physician evaluates the brain functions, related diseases, or even current emotion state of the patient with the brainwave patterns and variations recorded by a brainwave instrument. However, the conventional brainwave inspection technology may not give a certain inspection result instantly. The patient usually have to revisit the clinic for the inspection result. Therefore, it is the normal case of brainwave inspection currently: the patient cannot learn the status of his body instantly, and the physician is unlikely to undertake a corresponding treatment in situ.

Besides, the output of a brainwave inspection is usually presented in waveforms. However, the physician cannot learn the inspection result instinctively from the waveforms. The physician has to further undertake analysis to interpret the inspection result before telling the patient the state of his body. Once the physician is not sufficiently experienced in analyzing brainwave, the interpretation may be affected, and the clinical process may be decelerated.

### SUMMARY OF THE INVENTION

The primary objective of the present invention is solve the problem that the conventional brainwave inspection technology is unable to provide an instinctive diagnosis.

In order to achieve the abovementioned objective, the present invention provides a brainwave auscultation method, which comprises
Step I: providing a brainwave auscultation device, which includes a brainwave pickup unit, a signal processing unit connected with the brainwave pickup unit, and a loudspeaker unit connected with the signal processing unit;
Step II: acquiring a primitive brainwave signal of a testee by placing the brainwave pickup unit on the head of the testee, and transmitting the primitive brainwave signal to the signal processing unit; after acquiring the primitive brainwave signal, the signal processing unit filtering the primitive brainwave signal according to a waveband reservation standard to generate a preparatory signal, wherein the wavebands reserved by the waveband reservation standard include the δ waveband, the θ waveband, the α waveband, the β waveband, and the γ waveband; the signal processing unit shifting a central frequency of the preparatory signal to an audible range of human ears; the signal processing unit performing a spread-spectrum operation to the preparatory signal, whose central frequency has been shifted, to generate a pre-vocalization signal whose frequencies range from 20 Hz to 20 kHz; and
Step III: making the loudspeaker generate sounds based on the pre-vocalization signal.

In one embodiment, the signal processing unit shifts the central frequency to 1 kHz.

In one embodiment, Step 2 further includes a sub-step: using an amplifier circuit to amplify the primitive brainwave signal.

In one embodiment, the signal processing unit performs a spread-spectrum operation to each waveband of the preparatory signal according to a plurality of spread-spectrum ratios; each of the spread-spectrum ratios is effective to each of the wavebands of the preparatory signal.

In one embodiment, the spread-spectrum ratios are different.

In one embodiment, the signal processing unit performs a spread-spectrum operation to the preparatory signal based on a phase-locked-loop technology and a voltage-controlled oscillator modulation technology.

In one embodiment, the signal processing unit performs the spread-spectrum operation to the preparatory signal based on a phase-locked-loop technology, a voltage-controlled oscillator modulation technology, and at least one of an input frequency modulator technology, an output modulation technology, and a frequency divider modulation technology.

In comparison with the conventional technology, the present invention is characterized in presenting brainwaves in an audio way to enable physicians to learn the result of brainwave inspection instinctively. Besides, the present invention provides a vocalization technology to shift the central frequency of the frequency spectrum of the preparatory signal to an audible range of human ears, whereby the preparatory signal may be converted into a pre-vocalization signal that the loudspeaker can present.

### BRIEF DESCRIPTION OF THE DRAWINGS

FIG. 1 shows a flowchart of a brainwave auscultation method according to the first embodiment of the present invention.

FIG. 2 is a diagram schematically showing that the present invention uses a brainwave pickup unit to pick up brainwave signals according to one embodiment of the present invention.

FIG. 3 is a diagram schematically showing the structure of a brainwave auscultation device according to the first embodiment of the present invention.

FIG. 4 is a diagram schematically showing the structure of a brainwave auscultation device according to the second embodiment of the present invention.

FIG. 5 is a diagram schematically showing an embodiment of the present invention.

### DETAILED DESCRIPTION OF THE INVENTION

The present invention will be described in detail in cooperation with drawings below.

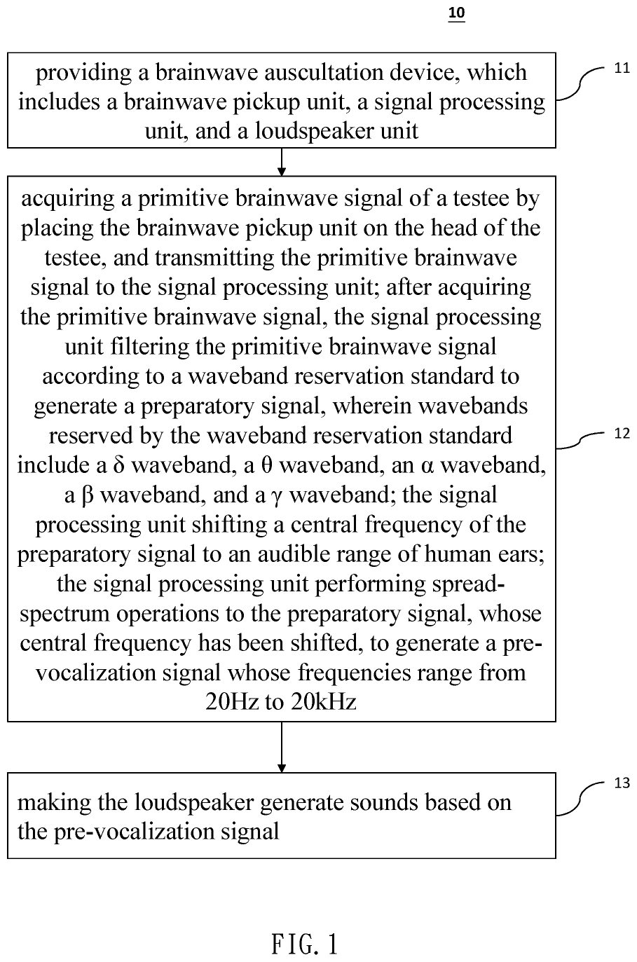

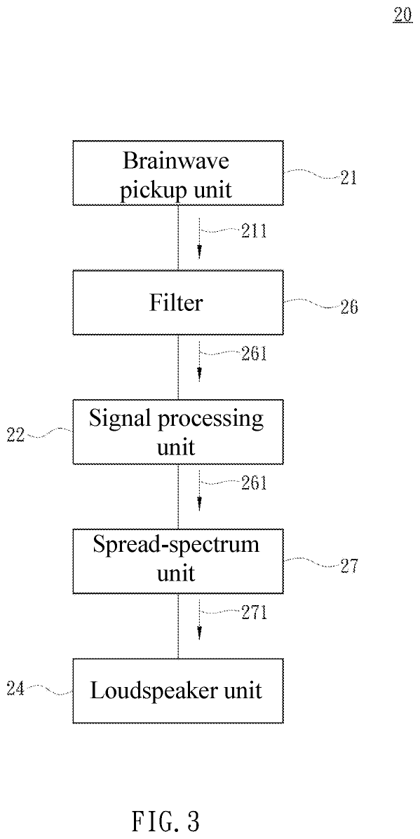

Refer to FIGS. 1-4. The present invention provides a brainwave auscultation method 10, which comprises
Step I (11): providing a brainwave auscultation device 20, which includes a brainwave pickup unit 21, a signal processing unit 22 connected with the brainwave pickup unit 21, and a loudspeaker unit 24 connected with the signal processing unit 22;

Step II (12): acquiring a primitive brainwave signal 211 of a testee 80 by placing the brainwave pickup unit 21 on the head of the testee 80, and transmitting the primitive brainwave signal 211 to the signal processing unit 22; after acquiring the primitive brainwave signal 211, the signal processing unit 22 filtering the primitive brainwave signal 211 according to a waveband reservation standard to generate a preparatory signal 261, wherein the wavebands reserved by the waveband reservation standard include a δ waveband, a θ waveband, an α waveband, a θ waveband, and a γ waveband; the signal processing unit 22 shifting a central frequency of the preparatory signal 261 to an audible range of human ears; the signal processing unit 22 performing a spread-spectrum operation to the preparatory signal 261, whose central frequency has been shifted, to generate a pre-vocalization signal 271 whose frequencies range from 20 Hz to 20 kHz; and
Step III (13): making the loudspeaker 24 generate sounds based on the pre-vocalization signal 271.

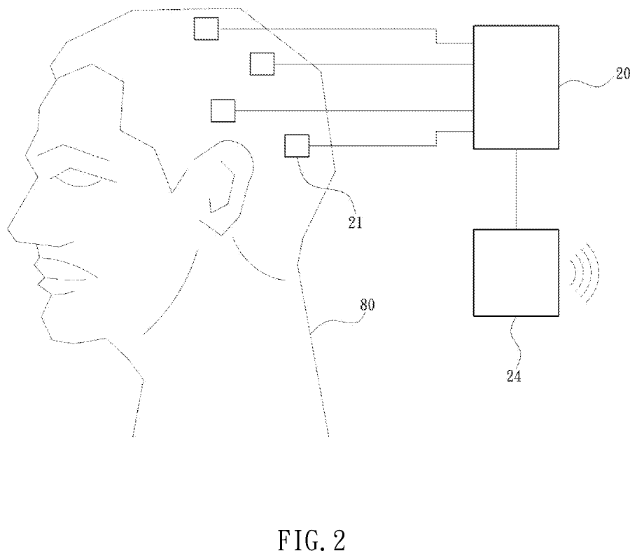

Refer to FIGS. 2-4 for detailed description. Firstly, the brainwave pickup unit 21 is placed on the head of the testee 80. The brainwave pickup unit 21 may include a plurality of patch-type electrodes that can be attached to the head of the testee 80. Alternatively, the brainwave pickup unit 21 may be a wearable device that can be worn on the head of the testee 80. The brainwave pickup unit 21 picks up the brainwave data of the testee 80 to acquire the primitive brainwave signal 211. The primitive brainwave signal 211 is very weak in practice. In one embodiment, after the brainwave pickup unit 21 acquires the primitive brainwave signal 211, an amplifier circuit 25 is used to amplify the primitive brainwave signal 211. Then, the amplified primitive brainwave signal 211 is transmitted to the signal processing unit 22. The primitive brainwave signal 211 acquired by the brainwave pickup unit 21 is an analog signal. However, the signals that can be processed by the signal processing unit 22 are digital signals. Thus, the primitive brainwave signal 211 needs to be converted from an analog signal to a digital signal before the brainwave pickup unit 21 transmits the primitive brainwave signal 211 to the signal processing unit 22, whereby the signal processing unit 22 can process the received signals.

Next, the signal processing unit 22 uses a filter 26 to perform a filtering treatment of the primitive brainwave signal 211. According to a waveband reservation standard, the filter 26 reserves a portion of wavebands of the primitive brainwave signal, including a δ waveband (0.15 Hz-3 Hz), a θ waveband (4 Hz-8 Hz), an α waveband (8 Hz-14 Hz), a β waveband (14 Hz-28 Hz), and a γ waveband (28 Hz-70 Hz). In other words, the filter 26 screens out the surges and noise of the primitive brainwave signal 211 outside the range of 0.15 Hz to 70 Hz and converts the primitive brainwave signal 211 into a preparatory signal 261 with the δ waveband, the θ waveband, the α waveband, the β waveband, and the γ waveband being reserved.

Further, the signal processing unit 22 uses the central frequency of the preparatory signal 261 as a datum point and shifts the central frequency to an audible range of human ears, such as the nearby of 1 kHz. Together with the central frequency, the other portions of the preparatory signal 261 are also shifted to other wavebands simultaneously, and the frequency differences between the central frequency and the other portions are kept. After shifting the preparatory signal 261, the signal processing unit 22 uses a spread-spectrum unit 27 to perform a spread-spectrum operation to the preparatory signal 26. The spread-spectrum unit 27 may be a portion of the signal processing unit 22 or an independent physical circuit. The signals processed by the spread-spectrum unit 27 are analog signals. If the spread-spectrum unit 27 is a physical circuit, the processing unit 22 has to convert the digital preparatory signal 261 into an analog signal before the signal processing unit 22 transmits the preparatory signal 261 to the spread-spectrum unit 27. After the spread-spectrum unit 27 receives the preparatory signal 261, the spread-spectrum unit 27 spreads the spectrum of the preparatory signal to a range between 20 Hz and 20 kHz and converts the preparatory signal 261 into a pre-vocalization signal 271 whose frequencies are in an audible range of human ears.

In Step III (13), the brainwave auscultation device 20 transmits the pre-vocalization signal 271 to the loudspeaker unit 24, and the loudspeaker 24 generates sounds based on the pre-vocalization signal 271. Thereby, the medical personnel can evaluate the status of the brainwave of the testee 80 according to the sounds generated by the loudspeaker unit 24.

Therefore, the present invention exempts physicians from reading brainwave data for diagnosis and enables physicians to diagnose the brainwave status of the testee 80 instinctively and conveniently in an audio way. Besides, the brainwave auscultation method 10 of the present invention shifts the central frequency of the preparatory signal 261 and spreads the frequency spectrum of the preparatory signal 261 to vocalize the primitive brainwave signal 211 through the loudspeaker unit 24.

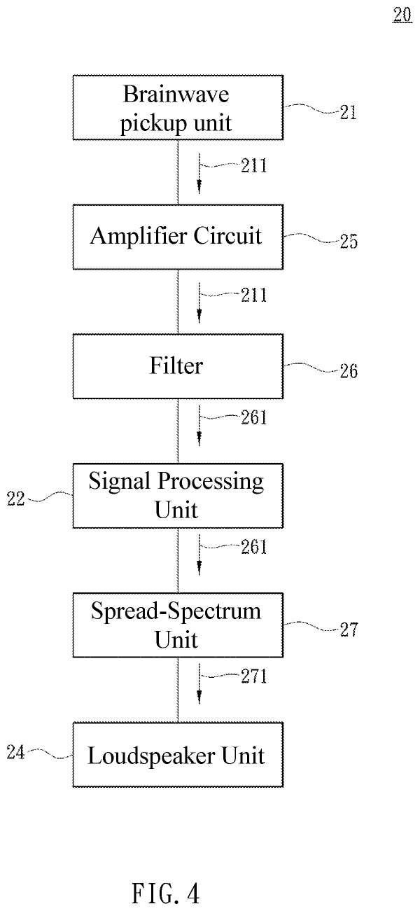

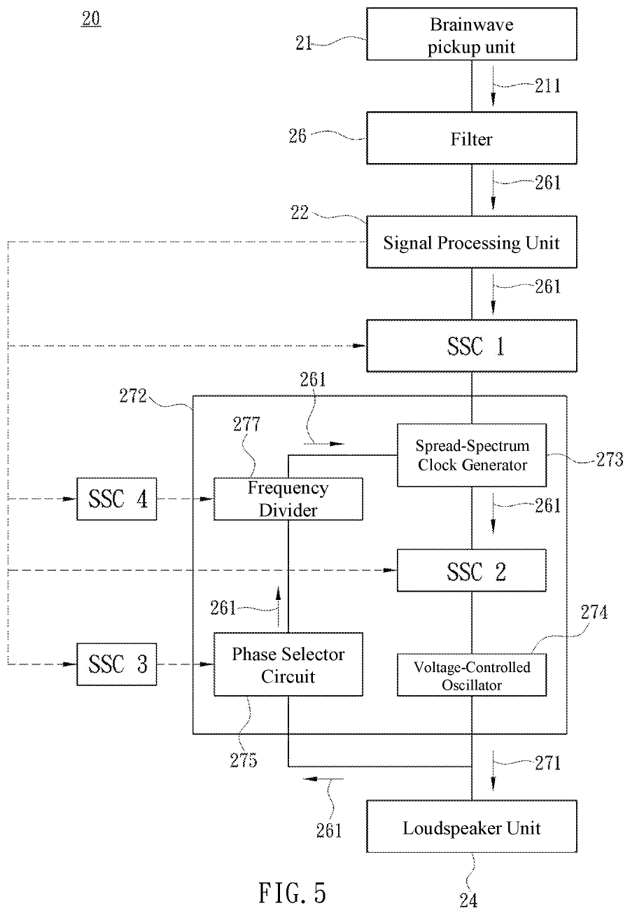

Refer to FIG. 4 and FIG. 5. In one embodiment, the signal processing unit 22 performs a spread-spectrum operation to the wavebands of the preparatory signal respectively according to a plurality of spread-spectrum ratios; the spread-spectrum ratios may be identical or different; each of the spread-spectrum ratios is effective to each of the wavebands of the preparatory signal. In detail, while the signal processing unit 22 performs the spread-spectrum operation, the spread-spectrum unit 27 spreads the spectrum using a phase-locked-loop technology and one of an input frequency modulator technology, a voltage-controlled oscillator modulation technology, an output modulation technology, and a frequency divider modulation technology. Refer to FIG. 5. In the phase-locked-loop technology, the spread-spectrum unit 27 uses a phase-locked loop 272 to realize the phase-locked-loop technology, wherein phase-locked loop 272 fixes the waveform of the preparatory signal 261 to fix the phase differences of the δ waveband, the θ waveband, the α waveband, the β waveband, and the γ waveband. In the input frequency modulator technology, after the phase-locked loop 272 has fixed the waveform of the preparatory signal 261, the spread-spectrum unit 27 uses a spread-spectrum clock generator 273 to modulate the frequency of the input preparatory signal 261, whereby the preparatory signal 261 is spread into the pre-vocalization signal 271. In the voltage-controlled oscillator modulation technology, the signal processing unit 22 uses a voltage-controlled oscillator 274 of the spread-spectrum unit 27 to realize the voltage-controlled oscillator modulation technology. After the phase-locked loop 272 has fixed the waveform of the preparatory signal 261, the voltage-controlled oscillator 274, succeeding to the spread-spectrum clock generator 273, modulates the voltage of the preparatory signal 261 to spread the frequency of the preparatory signal 261. In the embodiment, the input frequency modulator technology or the voltage-controlled oscillator modulation technology may respectively act on the wavebands with different spread-spectrum ratios. In the output modulation technology, the present invention may use a phase selector circuit 275 to realize the output modulation technology. After the phase-locked loop 272 has fixed the waveform of the preparatory signal 261, the phase selector circuit 275 delays the phase differences of the preparatory signal 261 to modulate the frequencies of the preparatory signal 261. In one embodiment, the present invention uses a delay circuit (not shown in the drawings) to realize the voltage-controlled oscillator modulation technology, as shown in FIG. 5. In one embodiment, the present invention uses a frequency divider 277 to realize the frequency divider modulation technology, wherein the frequency divider 277 uses an equation to modulate the frequencies of the preparatory signal 261, whereby to spread the frequency spectrum of the preparatory signal 261 with the waveform thereof being fixed. The equation is expressed as

$${f}_{o u t} = \frac{{f}_{i n}}{n} ,$$

wherein fₒᵤₜ is the post-modulation frequency of the preparatory signal 261, fᵢₙ is the pre-modulation frequency of the preparatory signal 261, and n is an integer.

While undertaking the spread-spectrum operation, the signal processing unit 22 may select different modes according to technologies provided by the spread-spectrum unit 27, such as SSC1-SSC4 shown in FIG. 5. In SSC1, the spread-spectrum unit 27 provides phase-locked-loop technology through the phase-locked loop 272, and then uses the spread-spectrum clock generator 273 and the voltage-controlled oscillator 274 to spread the spectrum of the preparatory signal 261. In SSC2, after fixing the phase differences of the wavebands of the preparatory signal 261, the spread-spectrum unit 27 uses the voltage-controlled oscillator 274 to spread the spectrum of the preparatory signal 261. In SSC3, the spread-spectrum unit 27 uses the phase-locked loop 272 in cooperation with the phase selector circuit 275 or the delay circuit to spread the spectrum, and then utilizes the frequency divider 277, the spread-spectrum clock generator 273, and the voltage-controlled oscillator 274. In SSC4, the spread-spectrum unit 27 provides phase-locked-loop technology through the phase-locked loop 272, and then utilizes the frequency divider 277, the spread-spectrum clock generator 273, and the voltage-controlled oscillator 274 to undertake spread-spectrum operation. It is learned from the above description: while performing the spread-spectrum technology, the spread-spectrum unit 27 may uses the locked-phase loop technology and the voltage-controlled oscillator modulation technology as the foundation in cooperation with the input frequency modulator technology and the output modulation technology/the frequency divider modulation technology. In order to transform the preparatory signal 261 into the pre-vocalization signal 271 whose frequency is in the audible range of human ears, the spread-spectrum unit 27 may select synergistic technologies according to requirement to realize the abovementioned target. Further, the spread-spectrum unit 27 may vary the cycles of the operations of the selected synergistic technologies. Therefore, the spread-spectrum ratio, which the spread-spectrum unit 27 instructs the voltage-controlled oscillator modulation technology and the synergistic technologies to set, may be varied.

The embodiments described above are only to exemplify the present invention but not to limit the scope of the present invention. Any equivalent modification or variation according to the spirit or claims of the present invention is to be also included by the present invention.

What is claimed is:

1. A brainwave auscultation method, comprising
Step I: providing a brainwave auscultation device, which includes a brainwave pickup unit, a signal processing unit connected with the brainwave pickup unit, and a loudspeaker unit connected with the signal processing unit;
Step II: acquiring a primitive brainwave signal of a testee by placing the brainwave pickup unit on the head of a testee, and transmitting the primitive brainwave signal to the signal processing unit; after acquiring the primitive brainwave signal, the signal processing unit filtering the primitive brainwave signal according to a waveband reservation standard to generate a preparatory signal, wherein wavebands reserved by the waveband reservation standard include a δ waveband, a θ waveband, an α waveband, a β waveband, and a γ waveband; the signal processing unit shifting a central frequency of the preparatory signal to an audible range of human ears; the signal processing unit performing spread-spectrum operation to the preparatory signal, whose central frequency has been shifted, to generate a pre-vocalization signal whose frequencies range from 20 Hz to 20 kHz; and
Step III: making the loudspeaker generate sounds based on the pre-vocalization signal.

2. The brainwave auscultation method according to claim 1, wherein the signal processing unit shifts the central frequency to 1 kHz.

3. The brainwave auscultation method according to claim 1, wherein in the Step II, after acquiring the primitive brainwave signal, the signal processing unit uses an amplifier circuit to amplify the primitive brainwave signal.

4. The brainwave auscultation method according to claim 1, wherein the signal processing unit performs the spread-spectrum operation to each waveband of the preparatory signal according to a plurality of spread-spectrum ratios; each of the spread-spectrum ratios is effective to each of the wavebands of the preparatory signal.

5. The brainwave auscultation method according to claim 4, wherein the spread-spectrum ratios are different.

6. The brainwave auscultation method according to claim 1, wherein the signal processing unit performs the spread-spectrum operation to the preparatory signal based on a phase-locked-loop technology and a voltage-controlled oscillator modulation technology.

7. The brainwave auscultation method according to claim 1, wherein the signal processing unit performs the spread-spectrum operation to the preparatory signal based on a phase-locked-loop technology, a voltage-controlled oscillator modulation technology, and at least one of synergistic technologies; the synergistic technologies include an input frequency modulator technology, an output modulation technology, and a frequency divider modulation technology.
